(19) United States
(12) Patent Application Publication
(10) Pub. No.: US 2023/0421869 A1
(43) Pub. Date: Dec. 28, 2023

(54) HAND-HELD, MULTI-BAND IMAGING PEN

(71) Applicant: MICRONVISION CORP., Bellevue, WA (US)

(72) Inventor: Xiaolong OUYANG, Bellevue, WA (US)

(21) Appl. No.: 18/211,486

(22) Filed: Jun. 19, 2023

Related U.S. Application Data
(63) Continuation-in-part of application No. 17/835,624, filed on Jun. 8, 2022, now Pat. No. 11,684,248.
Application No. 17/835,624 is a continuation-in-part of application No. 17/745,526, filed on May 16, 2022.
Application No. 17/745,526 is a continuation-in-part of application No. 17/473,587, filed on Sep. 13, 2021, now Pat. No. 11,330,973.
Application No. 17/473,587 is a continuation-in-part of application No. 17/362,043, filed on Jun. 29, 2021, now Pat. No. 11,350,816.
Application No. 17/362,043 is a continuation-in-part of application No. PCT/US19/36060, filed on Jun. 7, 2019.
Application No. PCT/US19/36060 is a continuation-in-part of application No. 16/363,209, filed on Mar. 25, 2019.
Application No. 16/363,209 is a continuation-in-part of application No. PCT/US17/53171, filed on Sep. 25, 2017.
(60) Provisional application No. 63/354,649, filed on Jun. 22, 2022.

(51) Int. Cl. H04N 23/13; H04N 23/56; H04N 23/51; H04N 23/11; A61B 1/06; A61B 1/04; A61B 1/00

(52) U.S. Cl. CPC H04N 23/13; H04N 23/56; H04N 23/51; H04N 23/11; A61B 1/0638; A61B 1/04; A61B 1/00016; A61B 1/0004; A61B 1/00045

(57) ABSTRACT

A hand-held, multi-band imaging pen is shaped and dimensioned for holding as a pen and has, at least at one end thereof, light sources and image sensors for imaging in white light and in non-white light, can have at the other end at least one light source and one light sensor, and can have a working channel for a medical instrument. The pen includes a wireless transmitter for sending images to an external display and tactile controls for lighting and camera functions.

### REFERENCE TO RELATED APPLICATIONS

This application claims the benefit of provisional application Ser. No. 63/354,649 filed Jun. 22, 2022 and is a continuation in-part of U.S. patent application Ser. No. 17/835,624 published as US 2022/0296090.

Ser. No. 17/835,624 is continuation in-part of Ser. No. 17/745,526 filed May 16, 2022.

Ser. No. 17/745,526 is a continuation in-part of Ser. No. 17/473,587 filed Sep. 13, 2021 and now U.S. Pat. No. 11,330,973.

Ser. No. 17/473,587 is a continuation in-part of each of: Ser. No. 17/362,043 filed Jun. 29, 2021, now U.S. Pat. No. 11,350,816, issued Jun. 7, 2022; International Patent Appl. No. PCT/US19/36060 filed Jun. 7, 2019; Ser. No. 16/363,209 filed Mar. 25, 2019 and published as US Pat. Appl. Publ. No. US2019/0216325; and International Patent Appl. No. PCT/US17/53171 filed Sep. 25, 2017.

This application incorporates by reference the entirety of the foregoing patent applications and claims the benefit of the filing date of each of the above-identified patent applications, as well as of the applications that they incorporated by reference, directly or indirectly, and the benefit of which they claim, including U.S. provisional applications, U.S. non-provisional applications, and international applications.

Said U.S. patent application Ser. No. 17/473,587 claims the benefit of and incorporates by reference each of the following provisional applications:
U.S. Prov. Ser. No. 63/218,362 filed Jul. 4, 2021
U.S. Prov. Ser. No. 63/213,499 filed Jun. 22, 2021
U.S. Prov. Ser. No. 63/210,034 filed Jun. 13, 2021
U.S. Prov. Ser. No. 63/197,639 filed Jun. 7, 2021
U.S. Prov. Ser. No. 63/197,611 filed Jun. 7, 2021
U.S. Prov. Ser. No. 63/183,151 filed May 3, 2021;
U.S. Prov. Ser. No. 63/153,252 filed Feb. 24, 2021;
U.S. Prov. Ser. No. 63/149,338 filed Feb. 14, 2021;
U.S. Prov. Ser. No. 63/138,751 filed Jan. 18, 2021;
U.S. Prov. Ser. No. 63/129,703 filed Dec. 23, 2020;
U.S. Prov. Ser. No. 63/124,803 filed Dec. 13, 2020;
U.S. Prov. Ser. No. 63/121,924 filed Dec. 6, 2020;
U.S. Prov. Ser. No. 63/121,246 filed Dec. 4, 2020;
U.S. Prov. Ser. No. 63/107,344 filed Oct. 29, 2020;
U.S. Prov. Ser. No. 63/087,935 filed Oct. 6, 2020;
U.S. Prov. Ser. No. 63/083,932 filed Sep. 27, 2020;
U.S. Prov. Ser. No. 63/077,675 filed Sep. 13, 2020; and
U.S. Prov. Ser. No. 63/077,635 filed Sep. 13, 2020.

This patent application is also related to and incorporates by reference each of the following international, non-provisional and provisional applications:
International Patent Application No. PCT/US17/53171 filed Sep. 25, 2017;
U.S. Pat. No. 8,702,594 Issued Apr. 22, 2014;
U.S. patent application Ser. No. 16/363,209 filed Mar. 25, 2019;
International Patent Application No. PCT/US19/36060 filed Jun. 7, 2019;
U.S. patent application Ser. No. 16/972,989 filed Dec. 7, 2020;
U.S. Prov. Ser. No. 62/816,366 filed Mar. 11, 2019;
U.S. Prov. Ser. No. 62/671,445 filed May 15, 2018;
U.S. Prov. Ser. No. 62/654,295 filed Apr. 6, 2018;
U.S. Prov. Ser. No. 62/647,817 filed Mar. 25, 2018;
U.S. Prov. Ser. No. 62/558,818 filed Sep. 14, 2017;
U.S. Prov. Ser. No. 62/550,581 filed Aug. 26, 2017;
U.S. Prov. Ser. No. 62/550,560 filed Aug. 25, 2017;
U.S. Prov. Ser. No. 62/550,188 filed Aug. 25, 2017;
U.S. Prov. Ser. No. 62/502,670 filed May 6, 2017;
U.S. Prov. Ser. No. 62/485,641 filed Apr. 14, 2017;
U.S. Prov. Ser. No. 62/485,454 filed Apr. 14, 2017;
U.S. Prov. Ser. No. 62/429,368 filed Dec. 2, 2016;
U.S. Prov. Ser. No. 62/428,018 filed Nov. 30, 2016;
U.S. Prov. Ser. No. 62/424,381 filed Nov. 18, 2016;
U.S. Prov. Ser. No. 62/423,213 filed Nov. 17, 2016;
U.S. Prov. Ser. No. 62/405,915 filed Oct. 8, 2016;
U.S. Prov. Ser. No. 62/399,712 filed Sep. 26, 2016;
U.S. Prov. Ser. No. 62/399,436 filed Sep. 25, 2016;
U.S. Prov. Ser. No. 62/399,429 filed Sep. 25, 2016;
U.S. Prov. Ser. No. 62/287,901 filed Jan. 28, 2016;
U.S. Prov. Ser. No. 62/279,784 filed Jan. 17, 2016;
U.S. Prov. Ser. No. 62/275,241 filed Jan. 6, 2016;
U.S. Prov. Ser. No. 62/275,222 filed Jan. 5, 2016;
U.S. Prov. Ser. No. 62/259,991 filed Nov. 25, 2015;
U.S. Prov. Ser. No. 62/254,718 filed Nov. 13, 2015;
U.S. Prov. Ser. No. 62/139,754 filed Mar. 29, 2015;
U.S. Prov. Ser. No. 62/120,316 filed Feb. 24, 2015; and
U.S. Prov. Ser. No. 62/119,521 filed Feb. 23, 2015.

### FIELD

This patent specification generally relates to imaging using a portable, hand-held device and more specifically a device that can be held like a pen and can image concurrently or sequentially in two or more different wavelength ranges.

### BACKGROUND

The immediate parent application describes endoscopy and colposcopy imaging of tissue such as the urinary and reproductive systems, including by multi-band imaging using endoscopes that comprise a single-use portion that is releasably coupled to a reusable portion to form the endoscope. The single-use portions typically are supplied in sterile packaging and contain light sources and image sensors. After a single-use portion and a reusable portion are assembled into an endoscope and used for a medical procedure, the single-use portion is discarded. Later, the reusable portion can be used with another sterile single-use portion for another medical procedure. The single-use portion can include light sources and image sensors operating in respective different wavelength ranges to produce separate or composite images for display on a screen that typically is mounted on the hand-held reusable portion.

This patent specification is directed to a different field in which an imaging device is pen-like and need not be discarded after a single use.

The subject matter described or claimed in this patent specification is not limited to embodiments that solve any specific disadvantages or that operate only in environments such as those described above. Rather, the above background is only provided to illustrate one exemplary technology area where some embodiments described herein may be practiced.

### SUMMARY

As described in the initially presented claims but subject to amendments thereof during prosecuting this patent application, according to some embodiments, a hand-held, multi-band imaging pen comprises: an elongated, pen-shaped housing that is shaped and dimensioned for holding as a pen and configured for directing a distal end thereof in or at a body opening or surface first light source selectively emitting white light from the housing to illuminate a first field of view (FOV); a second light source selectively emitting non-white light from housing to illuminate a second FOV; a first multi-pixel, two-dimensional (2D) image sensor in the housing, configured to image white light from said first FOV and produce white light image data; a second multi-pixel, 2D image sensor in the housing, configured to image non-white light from said second FOV and produce non-white light image data; a wireless transmitter in the housing, configured to receive said while light image data and non-white light image data and transmit the received image data; a display wirelessly coupled to the transmitter and configured to display image data transmitted thereto by the transmitter; a power source in said housing selectively powering said first and second light sources and first and second image sensors and said wireless transmitter; and a tactile control interface mounted to the housing and operatively coupled with said light sources and image sensors and wireless transmitter to control selective operation thereof.

According to some embodiments, the hand-held, multi-band imaging pen can further include one or more of the following features: (a) said first and second light sources can emit light from a single distal end of the housing; (b) said first and second FOVs can at least partly overlap; (c) the second light source can comprise a source of near infrared light (MR) and at least one of the first and second imaging sensors can comprise a light sensor having spatial resolution of at least 2000 pixels in at least one dimension; (d) the second light source can comprise a source of blue light and at least one of the first and second image sensors comprises a light sensor having spatial resolution of at least 2000 pixels in at least one dimension and configured to image blue light; (e) each of said first and second image data can have spatial resolution of at least 2000 pixels in at least one dimension; (f) said tactile control interface can comprise a finger-operate button mounted to and accessible from outside said housing; (g) said tactile control interface can comprise plural buttons mounted to and accessible from outside said housing and configured to operate respective functions of said light sources, image sensors and wireless transmitter; (h) the pen can further include a third light source emitting light from the housing to illuminate a third FOV and a third multi-pixel, 2D image sensor configured to image light from said third light source within said third FOV and produce third image data, and said wireless transmitter is further configured to receive said third image data and transmit the received third image date to the display; and (i) said first light source can be at one longitudinal end of the housing and said second light source can be is at an opposite longitudinal end of the housing.

According to some embodiments, a hand-held imaging pen comprises: an elongated, pen-shaped housing that is shaped and dimensioned for holding as a pen and configured for directing a distal end thereof in or at a body opening or surface; a first light source selectively emitting light in a first wavelength range from the housing to illuminate a first field of view (FOV); a first image sensor configured to image light in said first wavelength range from said first FOV and produce first image data; a wireless transmitter in the housing, configured to receive said first image data and transmit the received first image data; a display wirelessly coupled to the transmitter and configured to display image data transmitted thereto by the transmitter; a power source in said housing selectively powering said first light source and first image sensor and said wireless transmitter; and a tactile control interface mounted to the housing and operatively coupled with said first light source and sensor, and wireless transmitter for selective operation thereof.

According to some embodiments, the pen described in the immediately preceding paragraph can further comprise one or more of the following features: (a) said first wavelength range can correspond to white light; (b) said first wavelength range can correspond to infrared light; (c) said first selected wavelength range can correspond to blue light; (e) the pen can further include a second light source selectively emitting light from the housing in a second wavelength range different from the first wavelength range to illuminate a second FOV and a second image sensor configured to image light in said second wavelength range from said second FOV and produce second image data, wherein said transmitter is further configured to receive said second image data and transmit the received second image data to the display and said second light source and second image sensor are operatively coupled to said power source and control interface; (f) said tactile control interface can comprise plural buttons mounted to and accessible from outside said housing and configured to operate respective functions of said first and second light sources, first and second image sensors, and wireless transmitter; and (g) the first and second light sources can be at a single longitudinal end of the housing.

According to some embodiments, a method of imaging a field of view (FOV) within or at a body comprises: (a) illuminating the FOV with first and second light sources emitting light from an elongated, pen-shaped housing that is shaped and dimensioned for holding as a pen; (b) the first and second light sources emit light in respective first and second wavelengths ranges that differ from each other; (c) sensing light within said FOV and within the first and second wavelength ranges by respective first and second image sensors to produce respective multi-pixel, two-dimensional image data of light from the first and second light sources; (d) controlling operations of said first and second light sources and image sensors with buttons mounted to said housing; (e) wirelessly transmitting said image data to a display outside the housing; and (f) powering said light sources, image sensors, and wireless transmitter with a power source inside the housing.

According to some embodiments, the method described in the immediately preceding paragraph can further comprise the following features: (a) said first wavelength range can correspond to white light and said second wavelengths range can correspond to infrared light; and (b) said first and said second light sources can be at a single longitudinal end of said housing.

### BRIEF DESCRIPTION OF THE DRAWINGS

To further clarify the above and other advantages and features of the subject matter of this patent specification, specific examples of embodiments thereof are illustrated in the appended drawings. It should be appreciated that these drawings depict only illustrative embodiments and are therefore not to be considered limiting of the scope of this patent specification or the appended claims. The subject matter hereof will be described and explained with additional specificity and detail through the accompanying drawings in which:

FIG. 1 is a perspective view of a hand-held imaging pen being used to image an outer ear (pinna and/or ear canal) and to send image data to a remote display, according to some embodiments.

FIG. 2 is a perspective view of an imaging pen and a blow-up of a distal end thereof, according to some embodiments.

FIG. 3 is a front view of a distal end of an imaging pen that has two imaging modules each comprising a two light source and an image sensor, operating at respective different wavelength ranges, according to some embodiments.

FIG. 4 is a front view of a distal end of an imaging pen illustrating three sets each comprising a light source and an image sensor operating at respective wavelength ranges, according to some embodiments.

FIG. 5 is a side view of an imaging pen that has one or more light sources and imaging sensors operating at respective wavelength ranges at each of a distal and a proximal end of the pen, according to some embodiments.

FIG. 6 is a side view of an imaging pen that can be otherwise like pens in FIGS. 1-5 but shows more detail for a control interface, according to some embodiments.

FIG. 7 is a side view of an imaging pen that includes an internal channel and ports at each of its distal and proximal ends, according to some embodiments.

FIG. 8 is a front view of a distal end of an imaging pen that includes an internal channel and one or more light sources and image sensors, according to some embodiments.

FIG. 9 is a block diagram of functional components of an imaging pen, according to some embodiments.

### DETAILED DESCRIPTION

A detailed description of examples of preferred embodiments is provided below. While several embodiments are described, the new subject matter described in this patent specification is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all these details. Moreover, for the purpose of clarity, certain technical material that is known in the related art has not been described in detail to avoid unnecessarily obscuring the new subject matter described herein. It should be clear that individual features of one or several of the specific embodiments described herein can be used in combination with features of other described embodiments or with other features. Like reference numbers and designations in the various drawings indicate like elements.

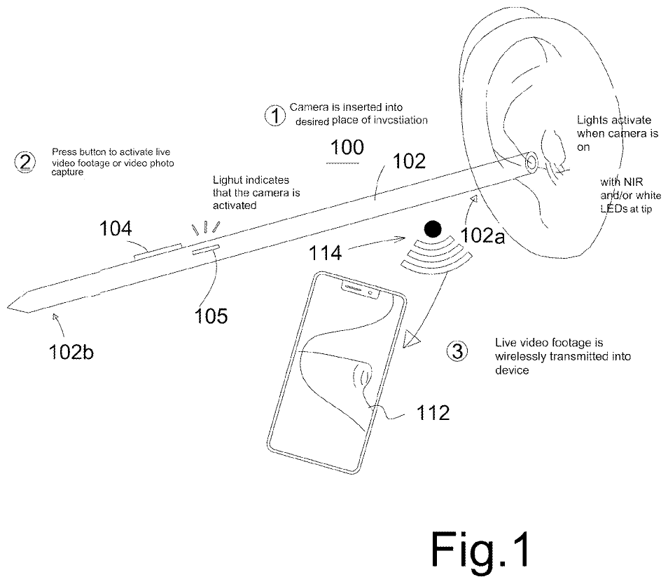

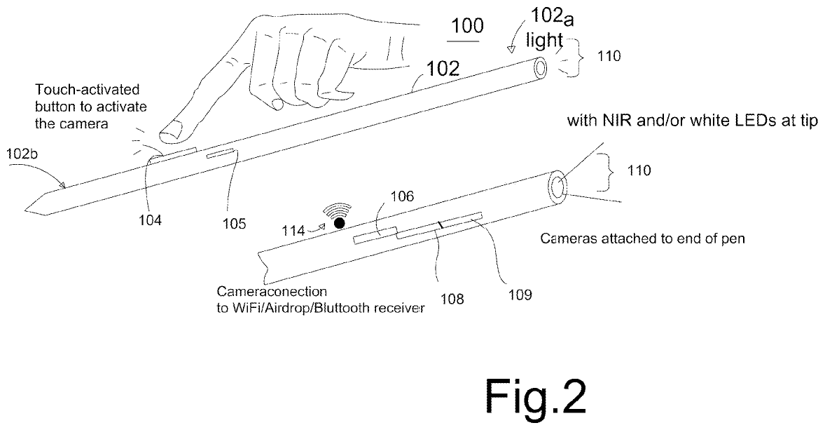

Referring to FIGS. 1-3, a hand-held imaging pen imaging pen 100 according to some embodiments comprises an elongated, tubular housing 102 that has one or more light sources and image sensors at a distal end 102a. Pen 100 includes a tactile control interface 104 that can comprise one or more finger-operated buttons and preferably has an indicator light that lights up when pen 100 is activated. Pen 100 includes an internal wireless transmitter 106 (FIG. 2) configured to wirelessly send image data acquired by an imaging module 110 at the distal end 102a of pen 100 to a display 112 that typically is physically separate from pen 100 and is configured to receive and process the image data and to display images. A processor 108 and a power source 109 such as a battery in housing 102 are coupled to components of pen 100 as discussed in more detail below. Display 112 can be a smart phone, a tablet, or some other device configured to wirelessly receive image data over a wireless link 114 such as WiFi or Bluetooth, process the received data for display, and display resulting images. An example of such device is display 150 described in said parent application published as US 202210296090.or external display 1414 described in U.S. patent application Ser. No. 17/720,143 hereby incorporated by reference. The image data transmitted to and displayed at display 112 can include video or still images.

FIG. 2 is a perspective view of imaging pen 100 and a partly sectional blow-up of its distal end 102a, according to some embodiments. Pen 100 of FIG. 2 is otherwise like pen 100 of FIG. 1 but is shown with internal components in housing 102, including wireless transmitter 106, processor 108, and power source 109 that are operatively coupled with control interface 104, light indictor 105, and imaging module 110 such that a user can activate pen 100 to take images with imaging module 110 and transmit them to display 112 by operating control interface 104.

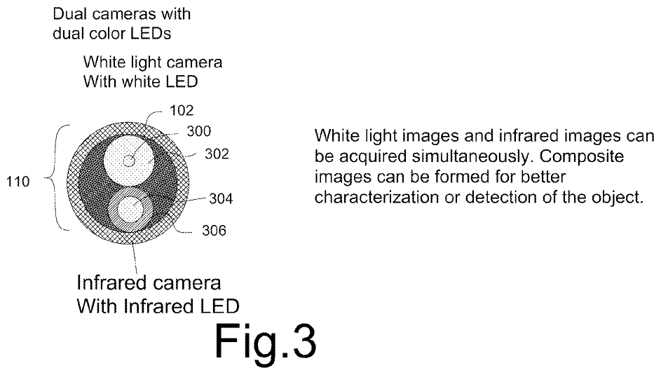

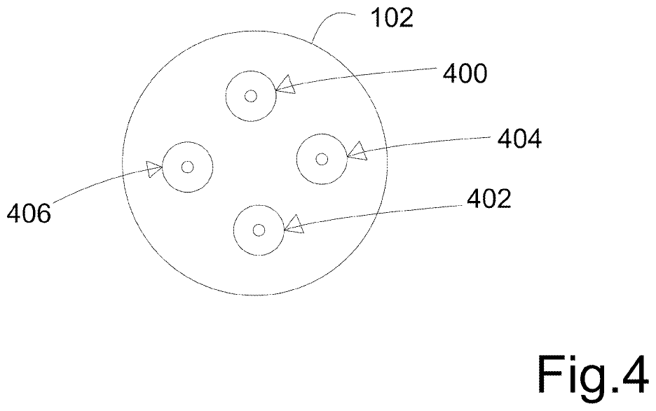

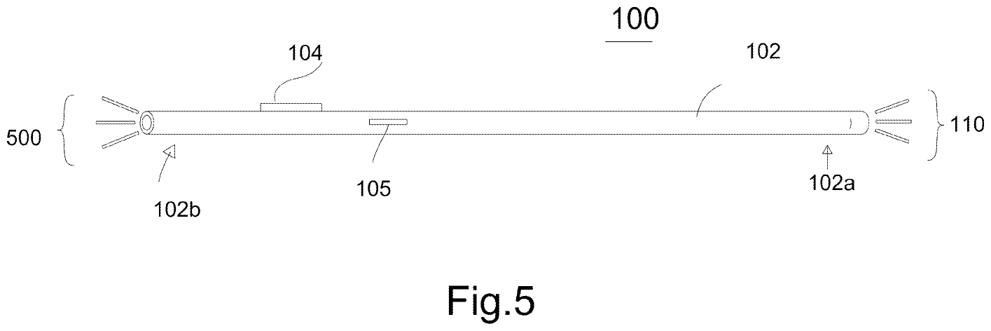

FIG. 3 is a front view of distal end 102a of imaging pen 100, which can be otherwise like pen 100 of FIGS. 1 and 2 but illustrates imaging module 110 in more detail, as comprising two sets each comprising a light source and an imaging sensor operating at respective wavelength ranges, according to some embodiments. In this example, imaging module 110 comprises a first image sensor 300 and associated first light source 302 operating in a first wavelength range and a second image sensor 304 and associated second light source 306 operating in a second wavelength range that preferably differs from the first wavelength range although in some embodiments this need not be the case. For example, the first light source 302 preferably emits white light and the second light source 306 preferably emits non-white light such as infrared light, or near infrared (NIR) light, or light in another non-white range such as blue light. Each of light sources 302 and 305 can comprise one or more LEDs emitting light in the desired wavelength range. The first image sensor 300 is adapted to image white light reflected within a first field of view (FOV) of sensor 300 and the second image sensor 304 is adapted to image the non-white range light emitted by the second light source 306 and reflected within a second FOV. The two FOVs can partly or fully overlap. The light sources and be LEDs described in said immediate parent application published as US 2022/0296090 and the image sensors can be small, 2-dimensional (2D) sensors, see for example FIGS. 4 and 5 thereof. The image sensors preferably have 2K spatial resolution (2,000 pixels in at least one dimension) and small area such as less than 3 mm in the largest dimension and are available from companies such as Omnivision, Sony and Samsung.

According to some definitions, white (or visible) light is in the range of 400-780 nm, infrared light is in the 780-10,000 nm wavelength range, NIR light is in the 780-2,500 nm range, and blue light is in the 440-490 nm range, but other generally accepted definitions specify somewhat different ranges. In a specific embodiment of pen 100, the second light source 306 emits NIR centered at approximately 940 nm.

FIG. 4 is otherwise like FIG. 3 but shows an imaging module 110 that comprises four sets of a light source and image sensor each. Each of sets 400, 402, 404 and 406 comprises a light source that can be one or more LEDs and an image sensor, and each is adapted to operate for a selected wavelength band. For example, sets 400 and 402 can be adapted for white light stereo imaging, and sets 404 and 406 can be dedicated to non-white light imaging. For example, both sets 404 can be for IR or NIR imaging, or both can be for blue light imaging, or one can be for IR imaging and the other for NIR imaging, or both can be for blue light imaging, or one can be for IR or NIR imaging and the other for blue light imaging. Three sets can be used in some embodiments and more sets in other embodiments.

FIG. 5 is a side view of an imaging pen 100 that has one or more light sources and image sensors operating at respective wavelength ranges at each of distal end 102a and proximal end 102b of imaging pen 100, according to some embodiments. Pen 100 of FIG. 5 is otherwise like pen 100 in FIGS. 1 and 2 but adds an imaging module 500 at the proximal end 102b of housing 102. Imaging module 500 can be any of the imaging modules described above for FIGS. 2-4.

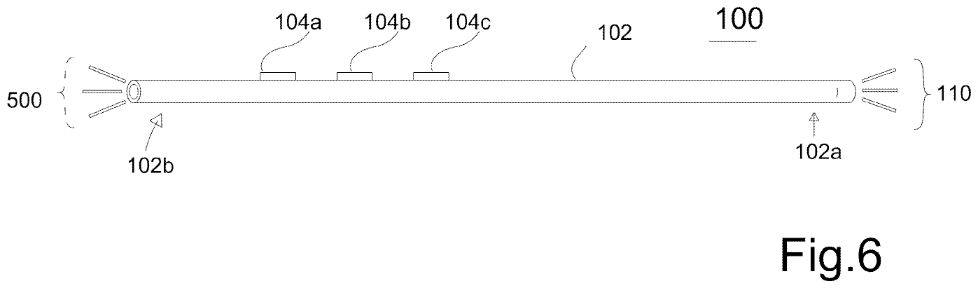

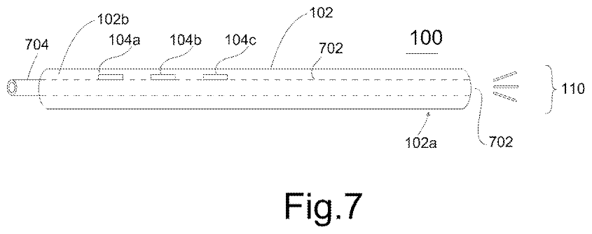

FIG. 6 is a side view of imaging pen 100 that can be otherwise like pens 100 discussed above for FIGS. 1-5 but shows more detail for control interface 104, according to some embodiments. In this example, tactile control interface 104 comprises plural buttons 104a, 104b, and 104c that can be configured to control respective operations of pen 100. For example, one of the buttons can be an ON-OFF button, another can operate one or some of the sets of a light source and an image sensor in imaging module 110 (and imaging module 500 if included in pen 100), and a third can operate some other function such as inflow/outflow discussed below for FIG. 7.

FIG. 7 is a side view of imaging pen 100 that includes an internal channel 700 and ports 702, 704 at the distal and proximal ends 102a, 102b, respectively, of housing 102, according to some embodiments. Channel 700 extends between ports 702 and 704 and can be used for flow in either direction of substances in liquid (or slurry), gas, and powder states. For example, a substance such as saline or medication can be pumped into port 704 using an external device such as a syringe and expelled through port 702 to thereby irrigate or medicate a body portion such as an ear or a nasal cavity while observing an image of the FOV(s) of imaging module 110 at display 112. As another example, suction can be applied to port 704 to thereby evacuate through port 704 material from a body portion while observing images within the FOV(s) of imaging module 110. As another example, a surgical instrument such as an injection needle or a grabber can be inserted through port 704 to protrude through port 702 to reach a desired site at a body surface or cavity.

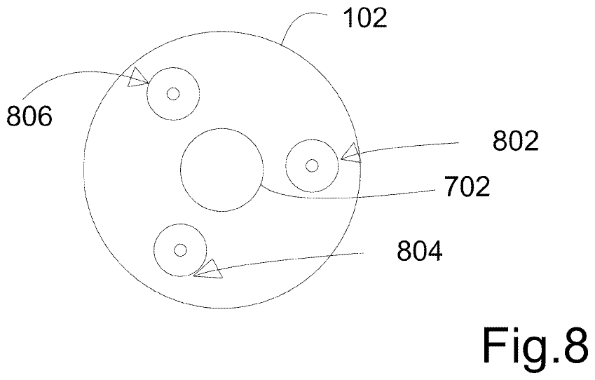

FIG. 8 is a front view of distal end 102a of imaging pen 100 of FIG. 7, according to some embodiments. As seen in FIG. 8, port 702 can be centrally located and circular but alternatively can be shaped differently and can be offset from the geometric center of housing 102. Distal end 102a of the example of FIG. 8 can include a single set 802 of a light source and image sensor, or an additional set 804, or more than two such sets, each operating for a selected wavelength range.

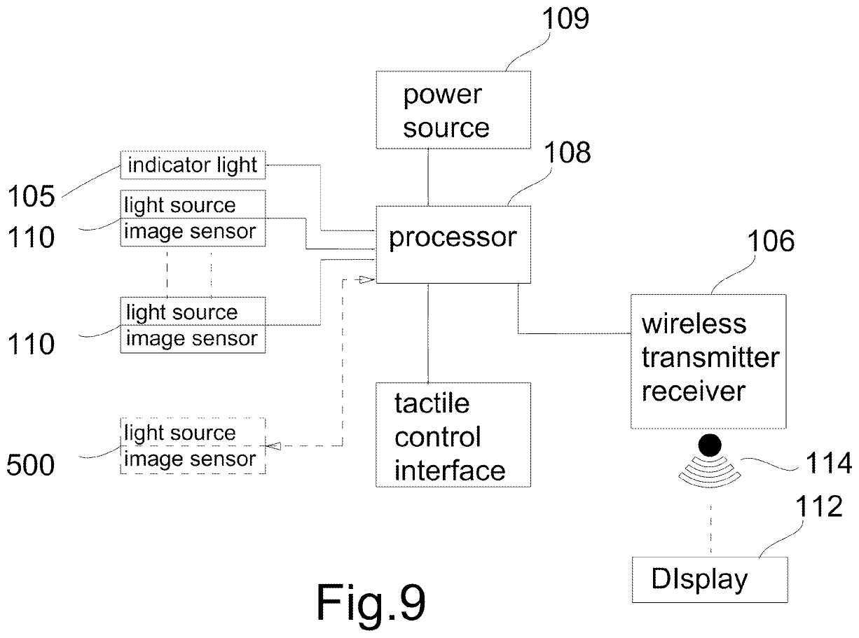

FIG. 9 is a block diagram of functional components of imaging pen 100, according to some embodiments. In operation, a user points distal end of pen 100 at a selected body service or inserts it in a body cavity such as the ear canal or a nostril and powers the pen by finger-operating control interface 104. Preferably, control interface 104 is configured to enable selection of any one of several modes of operation, for example to acquire and display both one or more white light images and one or more non-white light images, separately or superimposed for form a composite image, or only one or more white light images, or only one or more non-white images, and to select one or more wavelength band for any non-white image. If pen 100 has an imaging module at each longitudinal end, for example as imaging modules 110 and 500 in FIG. 5, the user can select which one to enable and points or inserts the enabled imaging module to image the desired site. Power source delivers power to the appropriate components as directed by processor 108. Processor 108 received image data from imaging module(s) 110 and, in some embodiments, from imaging module 500, and preferably applies some image processing to the received image data, such as formatting for wireless transmission or more sophisticated image processing. The output of processor 108 feeds transmitter 106 to deliver the image data to display 112. Preferably, display 112 further processes the received image data to improve the displayed images.

In some embodiments, transmitter 106 can be configured to two-way wireless communication and display 112 can be configured with a user interface such as a touch screen or a keyboard to enable a user to send commands via wireless link 114 to processor 108, for example to control the operation of imaging modules 106 and 500 as an addition or an alternative to commands that a user can enter through interface 104. In some embodiments, pen 100 need not include interface 104, i.e., there would no control buttons on the pen, so that all user interface commands can be provided through display 112. In some embodiments, one or more interface commands can be sent to processor 108 through one or more buttons 104 and some can be sent from display 112.

A typical pen 100 uses an imaging module 110 at its distal end 102a to image white light and non-white light such as NIR light that can be centered at 940 nm. When pen 100 has no internal channel, the outside diameter at distal end 102a preferably is less than 6 mm and more preferably is 4 mm or less and the distal face of distal end 102a is cylindrical and has rounded edges.

The imaging modules 1010 can be operated concurrently or in a selected time sequence so imaging pen 100 can produce and display images in the respective wavelength ranges concurrently or in a selected time sequence. These images can be displayed in a selected time sequence or can be composited, preferably in spatial registration, to produce and overlaid composite image in which the contribution of each wavelength range at a pixel can be a blend of two images in selected proportions that need not be the same, to thereby make features from one of the wavelength ranges stand out.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered as illustrative and not restrictive, and the body of work described herein is not to be limited to the details given herein, which may be modified within the scope and equivalents of the appended claims.

What it claimed is:

1. A hand-held, multi-band imaging pen comprising:
an elongated, pen-shaped housing that is shaped and dimensioned for holding as a pen and configured for directing a distal end thereof in or at a body opening or surface;
a first light source selectively emitting white light from the housing to illuminate a first field of view (FOV);
a second light source selectively emitting non-white light from housing to illuminate a second FOV;
a first multi-pixel, two-dimensional (2D) image sensor in the housing, configured to image white light from said first FOV and produce white light image data;
a second multi-pixel, 2D image sensor in the housing, configured to image non-white light from said second FOV and produce non-white light image data;
a wireless transmitter in the housing, configured to receive said while light image data and non-white light image data and transmit the received image data;
a display wirelessly coupled to the transmitter and configured to display image data transmitted thereto by the transmitter;
a power source in said housing selectively powering said first and second light sources and first and second image sensors and said wireless transmitter; and
a tactile control interface mounted to the housing and operatively coupled with said light sources and image sensors and wireless transmitter to control selective operation thereof.

2. The hand-held, multi-band imaging pen of claim 1, in which said first and second light sources emit light from a single distal end of the housing.

3. The hand-held, multi-band imaging pen of claim 2, in which said first and second FOVs at least partly overlap.

1. The hand-held, multi-band imaging pen of claim 1, in which the second light source comprises a source of near infrared light (NIR) and at least one of the first and second imaging sensors comprises a light sensor having spatial resolution of at least 2000 pixels in at least one dimension.

2. The hand-held, multi-band imaging pen of claim 1, in which the second light source comprises a source of blue light and at least one of the first and second image sensors comprises a light sensor having spatial resolution of at least 2000 pixels in at least one dimension and configured to image blue light.

3. The hand-held, multi-band imaging pen of claim 1, in which each of said first and second image data has spatial resolution of at least 2000 pixels in at least one dimension.

4. The hand-held, multi-band imaging pen of claim 1, in which said tactile control interface comprises a finger-operate button mounted to and accessible from outside said housing.

5. The hand-held, multi-band imaging pen of claim 1, in which said tactile control interface comprises plural buttons mounted to and accessible from outside said housing and configured to operate respective functions of said light sources, image sensors and wireless transmitter.

6. The hand-held, multi-band imaging pen of claim 1, further including a third light source emitting light from the housing to illuminate a third FOV and a third multi-pixel, 2D image sensor configured to image light from said third light source within said third FOV and produce third image data, and said wireless transmitter is further configured to receive said third image data and transmit the received third image date to the display.

7. The hand-held, multi-band imaging pen of claim 1, in which said first light source is at one longitudinal end of the housing and said second light source is at an opposite longitudinal end of the housing.

8. A hand-held imaging pen comprising:
an elongated, pen-shaped housing that is shaped and dimensioned for holding as a pen and configured for directing a distal end thereof in or at a body opening or surface;
a first light source selectively emitting light in a first wavelength range from the housing to illuminate a first field of view (FOV);
a first image sensor configured to image light in said first wavelength range from said first FOV and produce first image data;
a wireless transmitter in the housing, configured to receive said first image data and transmit the received first image data;
a display wirelessly coupled to the transmitter and configured to display image data transmitted thereto by the transmitter;
a power source in said housing selectively powering said first light source and first image sensor and said wireless transmitter; and
a tactile control interface mounted to the housing and operatively coupled with said first light source and sensor, and wireless transmitter for selective operation thereof.

9. The hand-held imaging pen as in claim 11, in which said first wavelength range corresponds to white light.

10. The hand-held imaging pen as in claim 11, in which said first wavelength range corresponds to infrared light.

11. The hand-held imaging pen as in claim 11, in which said first selected wavelength range corresponds to blue light.

12. The hand-held imaging pen as in claim 11, further including a second light source selectively emitting light from the housing in a second wavelength range different from the first wavelength range to illuminate a second FOV and a second image sensor configured to image light in said second wavelength range from said second FOV and produce second image data, wherein said transmitter is further configured to receive said second image data and transmit the received second image data to the display and said second light source and second image sensor are operatively coupled to said power source and control interface.

13. The hand-held imaging pen of claim 15, in which said tactile control interface comprises plural buttons mounted to and accessible from outside said housing and configured to operate respective functions of said first and second light sources, first and second image sensors, and wireless transmitter.

14. The hand-held imaging pen of claim 11, in which the first and second light sources are at a single longitudinal end of the housing.

15. A method of imaging a field of view (FOV) within or at a body, comprising:
illuminating the FOV with first and second light sources emitting light from an elongated, pen-shaped housing that is shaped and dimensioned for holding as a pen;
wherein the first and second light sources emit light in respective first and second wavelengths ranges that differ from each other;
sensing light within said FOV and within the first and second wavelength ranges by respective first and second image sensors to produce respective multi-pixel, two-dimensional image data of light from the first and second light sources;
controlling operations of said first and second light sources and image sensors with buttons mounted to said housing;
wirelessly transmitting said image data to a display outside the housing; and
powering said light sources, image sensors, and wireless transmitter with a power source inside the housing.

16. The method of claim 18, in which said first wavelength range corresponds to white light and said second wavelengths range corresponds to infrared light.

17. The method of claim 18, in which said first and said second light sources are at a single longitudinal end of said housing.
